(19) United States
(12) Patent Application Publication
(10) Pub. No.: US 2003/0108940 A1
(43) Pub. Date: Jun. 12, 2003

(54) Novel polymorphic microsatellite markers in the human MHC class II region

(75) Inventors: Hidetoshi Inoko, Atsugi-shi (JP); Gen Tamiya, Isehara-shi (JP); Yasunari Matsuzaka, Isehara-shi (JP)

Correspondence Address: JANIS K. FRASER, PH.D., J.D., Fish & Richardson P.C., 225 Franklin Street, Boston, MA, 02110-2804, US

(73) Assignee: Hidetoshi Inoko

(21) Appl. No.: 10/314,405

(22) Filed: Dec. 6, 2002

Related U.S. Application Data
(62) Division of application No. 09/713,616, filed on Nov. 15, 2000.

(51) Int. Cl. C12Q 1/68; C07H 21/04

(52) U.S. Cl. 435/6; 536/24.3

(57) ABSTRACT

Novel polymorphic microsatellite markers in the human MHC class II region and methods for disease mapping and genotyping with said microsatellite markers are provided. Said microsatellite markers are useful in HLA-related research, such as genetic mapping of HLA class II associated diseases, transplantation matching, population genetics, and identification of recombination hot spots as well as linkage disequilibrium studies.

### FIELD OF THE INVENTION

The present invention relates to novel polymorphic microsatellite markers in the human MHC class II region and methods for disease mapping and genotyping with said microsatellite markers.

### BACKGROUND OF THE INVENTION

The human major histocompatibility complex (MHC) is positioned on the short arm of the 6th chromosome, band p21.3, and has been divided into three non-overlapping segments called class I, II, and III (Campbell, D. and Trowsdale, J. (1997) Immunol. Today 18, 43; The MHC sequencing consortium (1999) Nature 401, 921-923). The HLA class II region can be largely subdivided into four subregions; DP, DO, DQ, and DR. The HLA class II genes display an extensive degree of genetic polymorphism and encode cell surface molecules that are involved in the presentation of exogenous antigens to the immune system (Kappes, D. and Strominger, J. L. (1988) Annu. Rev. Biochem. 57, 991-1028). Allelic variants of these class II genes are associated with a large number of diseases, e.g., rheumatoid arthritis (Shibue, T. et al. (2000) Arthritis and Rheumatism 43, 753-757), insulin-dependent diabetes mellitus (IDDM) (Sanjeevi, C. B. (2000) Human Immunology 61, 148-153; Todd, J. A. et al. (1987) Nature 329, 599-604; She, J.-X. (1996) Immunol. Today 17, 323-329), IgA deficiency (Olerup, O. et al. (1990) Nature 347, 289-290; Olerup, O. et al. (1992) Proc. Natl. Acad. Sci. USA 89, 10653-10657; Reil, A. et al. (1997) Tissue Antigens 50, 501-506), multiple sclerosis (Haegert, D. G. et al. (1989) J. Neurosci. Res. 23, 46-54; Haegert, D. G. and Francis, G. S. (1992) Hum. Immunol. 34, 85-90; Allen, M. et al. (1994) Hum. Immunol. 39, 41-48), idiopathic nephrotic syndrome (Konrad, M. et al. (1994) Tissue Antigens 43, 275-280), pemphigus vulgaris (Delgado, J. C. et al. (1996) Tissue Antigens 48, 668-672; Delgado, J. C. et al. (1997) Hum. Immunol. 57, 110-119), and idiopathic nonobstructive azoospermia (Tsujimura, A. et al. (1999) J. Androl. 20, 545-550).

To date, at least 197 HLA-DRB1, 19-DQA1, 35-DQB1, 13-DPA1, and 83-DPB1 alleles have been officially recognized (Marsh, S. G. E. (1998) Tissue Antigens 51, 467-507). The diversity of the HLA haplotypes in human populations has served as a useful landmark to roughly map disease-susceptibility loci in this region (Trowsdale, J. (1996) Molecular genetics of HLA class I and class II regions. In: Browning, M. J. and Mcmichael, A. J. eds., HLA and MHC genes, molecules and function. Oxford: Bios Scientific Publishers Ltd., 23-39; Hall, F. C. and Bowness, P. (1996) HLA and diseases: from molecular function to disease association? In: Browning, M. J. and Mcmichael, A. J. eds., HLA, and MHC genes, molecules and function. Oxford: Bios Scientific Publishers Ltd. 353-381). However, only five genes, encoding the polymorphic HLA antigens (HLA-DRB1, -DQA1, -DQB1, -DPA1, and -DPB1), have so far been available as genetic markers in this region. Given this and the fact that the region spans over approximately 1.1 Mb and contains more than 30 functional genes (Forbes, S. A. and Trowsdale, J. (1999) Immunogenetics 50, 152-159; Beck, S. and Trowsdale, J. (1999) Immunological Reviews 167, 201-210), it is therefore difficult, if not impossible, to precisely pinpoint most of disease susceptibility loci to their respective single genetic entities using only the available HLA class II diversity.

This is mainly because of the tight linkage disequilibrium observed throughout the class II region. For example, IDDM was first reported to be associated with DR3 and DR4 in Caucasoids (Rotter, J. I. et al. (1983) Diabetes 32, 169-174). Since then, many studies using world-wide populations have shown associations not only with DRB1, but also with DQA1, DQB1, and DPB1 alleles (Thompson, G. et al. (1988) Am. J. Hum. Genet. 43, 799; Rønningen, K. S. et al. (1992) HLA class II associations in insulin dependent diabetes mellitus among Blacks, Caucasoids and Japanese. In: Tsuji, K. et al. eds., HLA 1991, vol.1. Oxford: Oxford University Press; Caillat-Zucman, S. et al. (1997) Insulin dependent diabetes mellitus (IDDM): 12th International Histocompatibility Workshop study. In: Charron, D. ed., HLA, vol. 1. France: EDK). The highest risk for developing the disease has been associated with the heterozygous DR3/DR4 phenotype, particularly in combination with DQA1*0501-DQB1*0201/DQA1*0301-DQB1*0302 alleles in Caucasian populations (Owerbach, D. et al. (1983) Nature 303, 815-817; Arnheim. N et al. (1983) Proc. Natl. Acad. Sci. USA 82, 6970-6974; Cohe-Haguenauer, O. et al. (1985) Proc. Natl. Acad. Sci. USA 82, 3335-3339; Bohme, J. et al. (1986) J. Immunol. 137, 941-947; Festenstein, H. et al. (1986) Nature 322, 64-67; Nepom, B. S. et al. (1986) J. Exp. Med. 164, 345-350; Schreuder, G. M. et al. (1986) J. Exp. Med. 164, 938-943; Tait, B. D. and Boyle, A. J. (1986) Tissue Antigens 28, 65-71), suggesting that HLA-DQ rather than -DR is involved in genetic predisposition to IDDM (57 DQB1 non-Asp theory) (Todd, J. A. et al. (1987) Nature 329, 599-604). However, transracial studies have revealed that the susceptible molecules and the degree of their respective contribution appear to be different in various populations. In Chinese and Japanese, for instance, the DR3/DR4 heterozygous allele and the DR4 homozygous allele, respectively, are strong susceptible genotypes (Hu, C. Y. et al. (1993) Hum. Immunol. 38, 105-114; Huang, H. S. et al. (1988) J. Formosan Med. Assoc. 87, 1-6; Huang, H. S. et al. (1992) J. Formosan Med. Assoc. 91, 233-236; Ju, L. Y. et al. (1991) Tissue Antigens 37, 218-223). Thus, it has been difficult to determine which one, DR or DQ locus, is the true pathogenic gene for IDDM.

Microsatellites are tandemly repeated sequences of 2˜6 bps which are widely dispersed throughout the human genome (Amos, W. and Rubinsztein, D. C. (1996) Nature genetics 12, 13-14; Edwards, A. I. et al. (1991) Am. J. Hum. Genet. 49, 746-756). They have been extensively used for linkage mapping as well as forensic and population studies (Bowcock, A. M. et al. (1994) Nature 368, 455-457; Brinkmann, B. et al. (1996) Hum. Genet. 98, 60-64). Polymorphism observed at these loci is due simply to variation in the number of repeats of a single unit; the so-called stepwise model. Valdes, A. M. et al. (1993) Genetics 133, 737-749; Levinson, G. and Gutman, G. A. (1987) Mol. Biol. Evol. 4, 203-221).

Previously, 38 polymorphic microsatellite repeats in the HLA class I region were collected (Tamiya, G. et al. (1998) Tissue Antigens 51, 337-346; Tamiya, G. et al. (1999) Tissue Antigens 54, 221-228). These microsatellites were subsequently used for association mapping of HLA class I associated diseases leading, among others, to a successful narrowing of critical regions for Behcet's disease and psoriasis vulgaris to approximately 50 kb between the MICA and HLA-B genes, and between the POU5F1 and S genes, respectively (Ota, M. et al. (1999) Am. J. Hum. Genet. 64, 1406-1410; Oka, A. et al. (1999) Hum. Mol. Genet. 88, 2165-2170).

In the HLA class II region, however, no polymorphic microsatellite repeat has been identified yet. Therefore, it is still unavailable to map susceptibility genes for diseases associated with HLA class II alleles.

### SUMMARY OF THE INVENTION

An objective of the present invention is to provide novel polymorphic microsatellite markers in the human MHC class II region and methods for disease mapping and genotyping with said microsatellite markers. These novel polymorphic microsatellites will provide useful genetic markers in HLA-related research, such as genetic mapping of HLA class II associated diseases, transplantation matching, population genetics, and identification of recombination hot spots as well as linkage disequilibrium studies.

The present inventors have analyzed 2˜5 base short tandem repeats (microsatellites) in the genomic sequence of the HLA class II region (1.1 Mb) by the computer program of Abajian (http://www.abajian.com/sputnik/) to identify a total number of 494 microsatellites from the genomic sequence. From among them, the present inventors selected microsatellites with more than 10 repeats for di-nucleotide repeats and with more than 5 repeats for tri-, tetra-, and penta-nucleotide repeats to obtain 145 microsatellites.

The present inventor, then, randomly chosen 41 out of the 145 microsatellite repeats mentioned above and predicted, by a rough survey using 8 Japanese HLA homozygous B-cell lines, that 31 out of these 41 microsatellite repeats should be quite polymorphic. Furthermore, the present inventors investigated allele frequencies and heterozygosities of 21 out of these 31 microsatellite repeats using 190 unrelated Japanese individuals by the polymerase chain reaction (PCR) combined with fluorescent-based automated fragment technology. Finally, the present inventors obtained 21 novel polymorphic microsatellite markers with high number of alleles and high polymorphism information content (PIC) to accomplish the present invention.

Namely, the present invention relates to novel polymorphic microsatellite markers in the MHC HLA class II region and methods for disease mapping and genotyping with said microsatellite markers. More specifically, the present invention relates to:

1) An oligonucleotide primer, wherein said primer is capable of specifically hybridizing to a DNA having the sequence of the flanking regions of a microsatellite selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48.

2) The oligonucleotide primer according to 1), wherein the sequence of said primer is selected from the group consisting of SEQ ID NOs: 1-42.

3) A kit for determining the number of repeat units of a microsatellite selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48, the kit comprising a pair of oligonucleotide primers having the sequence of the flanking regions of said microsatellite.

4) The kit according to 3), comprising a pair of oligonucleotide primers selected from the group consisting of

(a) SEQ ID NO: 1 and SEQ ID NO: 2,

(b) SEQ ID NO: 3 and SEQ ID NO: 4,

(c) SEQ ID NO: 5 and SEQ ID NO: 6,

(d) SEQ ID NO: 7 and SEQ ID NO: 8,

(e) SEQ ID NO: 9 and SEQ ID NO: 10,

(f) SEQ ID NO: 11 and SEQ ID NO: 12,

(g) SEQ ID NO: 13 and SEQ ID NO: 14,

(h) SEQ ID NO: 15 and SEQ ID NO: 16,

(i) SEQ ID NO: 17 and SEQ ID NO: 18,

(j) SEQ ID NO: 19 and SEQ ID NO: 20,

(k) SEQ ID NO: 21 and SEQ ID NO: 22,

(l) SEQ ID NO: 23 and SEQ ID NO: 24,

(m) SEQ ID NO: 25 and SEQ ID NO: 26,

(n) SEQ ID NO: 27 and SEQ ID NO: 28,

(o) SEQ ID NO: 29 and SEQ ID NO: 30,

(p) SEQ ID NO: 31 and SEQ ID NO: 32,

(q) SEQ ID NO: 33 and SEQ ID NO: 34,

(r) SEQ ID NO: 35 and SEQ ID NO: 36,

(s) SEQ ID NO: 37 and SEQ ID NO: 38,

(t) SEQ ID NO: 39 and SEQ ID NO: 40, and

(u) SEQ ID NO: 41 and SEQ ID NO: 42.

5) A method for determining the number of repeat units of a microsatellite, the method comprising a step for determining the number of repeat units in the region of which DNA can be amplified by using the kit according to 4).

6) A method for mapping of susceptibility genes for disease associated with HLA class II alleles, by using a microsatellite marker selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48, the method comprising:

(a) determining the number of repeat units of said microsatellite,

(b) estimating the allele frequencies of patients and controls, based on said number, and

(c) comparing the allele frequencies of patients with those of controls.

7) The method according to 6), the method comprising:

(a) amplifying a region of microsatellite using the oligonucleotide primer according to 1) or 2),

(b) determining the number of repeat units of said microsatellite,

(c) estimating the allele frequencies of patients and controls, based on the number, and

(d) comparing the allele frequencies of patients with those of controls.

8) A method for genotyping of a microsatellite allele selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48, the method comprising:

(a) amplifying a region of the microsatellite, and

(b) determining the number of repeat units of said microsatellite.

9) The method according to 7), wherein said amplifying is performed by using the oligonucleotide primer according to 1) or 2).

### BRIEF DESCRIPTION OF THE DRAWINGS

FIG. 1 shows the gene map and location of microsatellite repeats in the HLA class II region. The top line indicates the scale of the entire HLA region and the map position of representative HLA antigen genes, HLA-DP, -DQ, -DR, -B, -C, -E, -J, -A, -G, and -F. Rectangles on the second line indicate the already known genes in the HLA class II region. Arrows show the transcriptional orientation of these genes. The bottom line indicates the location of the polymorphic microsatellite markers of the present invention.

### DETAILED DESCRIPTION OF THE INVENTION

In the present invention, the term “microsatellite” means a 2˜5 base short tandem repeat in a polynucleotide sequence. The microsatellites are classified into the following three kinds of repeats: perfect repeat, imperfect repeat, and compound repeat. A perfect repeat is defined as a tandem repeat without interruption and without adjacent repeats of another sequence. An imperfect repeat is defined as two or more runs of uninterrupted repeats separated by nonrepeat bases. A compound repeat corresponds to those containing stretches of two or more different repeats. Preferably, the microsatellite of the present invention is a perfect or imperfect repeat.

The microsatellite of the present invention is named as “M2_n_m”, where “M2” represents the serial number of temporary consensus genomic sequence, “n” indicates the number of nucleotides in repetition units (2˜5), and “m” represents a serial number.

Microsatellite loci that are useful in the present invention will have the general formula:

L(M)ₙR

where L and R are non-repetitive flanking sequences that uniquely identify the particular locus, M is a repeat motif, and n is the number of repeats. The locus may be present inside or outside coding region of genes on a human chromosome.

The flanking sequences L and R uniquely identify the microsatellite locus within the human genome. L and R will be at least about 18 nucleotides in length, and may extend a distance of several thousand bases. The DNA having L and R sequences may be obtained in substantial purity as restriction fragments, amplification products, etc., and will be obtained as a sequence other than a sequence of an intact chromosome. Usually, the DNA will be obtained substantially free of other nucleic acid compounds which do not include a microsatellite sequence or fragment thereof, generally being at least about 50%, usually at least about 90% pure and are typically “recombinant”, i.e., flanked by one or more nucleotides with which they are not normally associated on a natural chromosome.

Within the flanking sequences L and R, sequences will be selected for amplification primers P_{L} and P_{R}. The exact compositions of the primer sequences are not critical to the invention, but P_{L} and P_{R} must hybridize to the flanking sequences L and R respectively, or complementary strand thereof, under stringent conditions. Conditions for stringent hybridization are known in the art, for example, one may use a solution of 5×SSC and 50% formamide, incubated at 42° C., preferably 50° C. or 65° C. To maximize the resolution of size differences at the locus, it is preferable to chose a primer sequence that is close to the repeat sequence, usually within at least about 100 nucleotides of the repeat, more usually at least about 50 nucleotides, and preferably at least about 25 nucleotides. Algorithms for the selection of primer sequences are generally known, and are available in commercial software packages. The primers will hybridize to complementary strands of chromosomal DNA, and will prime towards the repeat sequences, so that the repeats will be amplified. The primers will usually be at least about 18 nucleotides in length, and usually not more than about 35 nucleotides in length. Primers may be chemically synthesized in accordance with conventional methods or isolated as fragments by restriction enzyme digestion, etc.

The term “polymorphic” means that, n, the number of repeat motif M at a specific locus, is variable in a population. Therefore, the polymorphisms of a microsatellite are represented as the differences in the length of DNA that lies between the flanking sequences L and R. The differences can be detected by amplifying a region of the microsatellite using suitable primers, size fractionating the amplified products by a denaturing polyacrylamide gel electrophoresis, and comparing the size of the amplified products. It is expected that microsatellites with more than 10 repeats for di-nucleotide repeats and with more than 5 repeats for tri-, tetra-, and penta-nucleotide repeats display a high degree of repeat polymorphism (Weber, W. J. L. (1990) Genomics 7, 524-530).

When the observed frequencies of i th and j th alleles at a given microsatellite locus are represented as pᵢ and pⱼ, heterozygosity (Ht) is calculated with:

Ht=1−Σpᵢ²

and polymorphism information content (PIC) value is calculated with:

PIC=Ht−ΣΣ2pᵢ²pⱼ².

Higher Ht and PIC values in the population reflect the higher degree of variability within the locus. In the present invention, the Ht value is preferably at least 0.5 and is more preferably at least 0.7, and PIC value is preferably at least 0.25 and is more preferably at least 0.5.

The present invention relates to oligonucleotide primers capable of specifically hybridizing to the flanking regions of the following 21 microsatellites:

| | | |
|---|---|---|
| M2_4_9 | M2_2_23 | M2_4_33 |
| M2_2_9 | M2_2_24 | M2_4_37 |
| M2_2_12 | M2_4_25 | M2_3_22 |
| M2_3_11 | M2_4_26 | M2_2_36 |
| M2_2_20 | M2_2_29 | M2_5_11 |
| M2_2_21 | M2_2_32 | M2_2_46, and |
| M2_2_22 | M2_4_32 | M2_2_48 |

“Specifically hybridizing” means that there is no significant cross-hybridization to unrelated regions of the genome under an ordinary hybridization conditions, and preferably under a stringent hybridization conditions. The microsatellites of the present invention comprises the respective repeat units indicated in Table 3. “Flanking regions of a microsatellite” are regions located upstream and downstream of each repeat unit, which is in between the two regions. Namely, the above-mentioned microsatellite is defined as a region comprising the repeat unit shown in Table 3 and existing in the genome in between the flanking regions indicated in Table 3. It is to be noted that, in Table 3, the antisense-strand nucleotide sequences of the flanking regions are indicated as 5′-3′ direction. The oligonucleotide primer of the present invention comprises a nucleotide sequence complementary to the sequence of either of the flanking region, or the complementary strand thereof; and preferably the primer has 18 nucleotide residues or more. The term “complementary strand” here indicates opposite strand to one strand of a DNA duplex consisting of A/T (U in the case of RNA) and G/C base pairs. “Complementary” means not merely being fully complementary in the region with at least 18 consecutive nucleotides but also being homologous in at least 70% of the nucleotides, preferably in at least 80% of the nucleotides, more preferably in at least 90%, yet more preferably in 95% or more of the nucleotides. Nucleotide sequence homology is determined by using a publicly known algorithm such as BLASTIN. Preferable nucleotide sequences of the oligonucleotide primers of the present invention are shown in SEQ ID NOs: 1-42. The relation between each SEQ ID NO and the microsatellite sequence is indicated in Table 3. Each nucleotide sequence of SEQ ID NOs: 1-42 is just an example; and the oligonucleotide primers of the present invention should be construed as not to be limited to the nucleotide sequences illustrated. Therefore, the oligonucleotide primers of the present invention include any oligonucleotide primers capable of amplifying regions containing the full-length repeat units amplified by using the oligonucleotide primers indicated in Table 3. The number of repeat units consisting microsatellites can be determined by amplifying the repeat units with the oligonucleotide primers of the present invention.

Any suitable amplification procedure known to one skilled in the art, such as, but not limited to, polymerase chain reaction (PCR), Qβ replication, isothermal sequence replication, or ligase chain reaction may be used. However, the most developed and well understood amplification systems are PCR systems. Thus, PCR is currently the preferred method of amplification. Suitable reaction conditions for PCR are described in Saiki et al. (1985) Science 239, 487, and Sambrook, et al. (1989) Molecular Cloning: A Laboratory Manual, Cold Spring Harbor Laboratory Press, 14.2-14.33.

Conveniently, a detectable label will be included in the amplification reaction. Suitable labels include fluorochromes, e.g., fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescrin (JOE), 6-carboxy-X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), 6-carboxy-4,7,2′,7′-tetrachloro-fluorescein (TET), or New Electrophoresis Dye (NED); radioactive labels, e.g., 32P, 35S, 3H; etc. The label may be a two-stage system, where the amplified DNA is conjugated to biotin, haptens, etc., having a high affinity binding partner, e.g., avidin, specific antibodies, etc., where the binding partner is conjugated to a detectable label. The label may be conjugated to one or both of the primers. Alternatively, the pool of nucleotides used in the amplification may be labeled, so as to incorporate the label into the amplification product.

Detection and size determination of amplification products such as PCR products from a specific microsatellite locus can be accomplished by several means. In one embodiment, amplification products are labeled with ³²P, size fractionated by a denaturing polyacrylamide gel electrophoresis, and visualized by autoradiography. In another embodiment, the amplification products are labeled with a fluorochrome, and separated on an automated DNA sequencing apparatus. The automated sequencer is particularly useful with multiplex amplification. Another method separates the amplification products by capillary electrophoresis, which has the advantage of being much faster than acrylamide gel electrophoresis while maintaining the accuracy of sizing. A review of capillary electrophoresis may be found in Landers et al. (1993) BioTechniques 14, 98-111.

Simultaneous analysis can be performed for multiple, different type microsatellites in the present invention. To achieve this, respective primer sets for amplifying multiple microsatellites are pre-labeled with different labels. The resulting amplification products obtained are fractionated, for example, by capillary electrophoresis, and lengths of the fragments are determined for each label, thereby achieving the simultaneous analysis of multiple, different type microsatellites.

The size of the amplification product is proportional to n, the number of repeats that are present at the locus specified by the primers. The size will be polymorphic in the population, and is therefore an allelic marker for that locus.

A kit may be provided for practice of the present invention. Such a kit will contain at least one set of oligonucleotide primers of present invention, useful for amplifying microsatellite DNA repeats. The primers may be conjugated to a detectable label.

The present invention also relates to a method for genotyping comprising the following steps (a) and (b):

(a) amplifying a region of the microsatellite, and

(b) determining the number of repeat units of said microsatellite.

The present invention further relates to a method for mapping of susceptibility genes for disease associated with HLA class II alleles, comprising the following step (c):

(c) estimating the allele frequencies of patients and controls, based on the number, and the method of the present invention preferably comprises the following step (d):

(d) comparing the allele frequencies of patients with those of controls.

DNA corresponding to a region of the microsatellite is amplified from a human genomic DNA sample by a publicly known method. The above-mentioned microsatellite can be amplified with the oligonucleotide primers of the present invention by using total genomic DNA or purified DNA containing the HLA class II region as a template. The number of repeat units in the amplification products is determined according to the method as described above. The number of repeat units of a microsatellite represents a genotype of the individual from which the genomic DNA has been derived.

Analysis for establishing a link between the thus determined genotype and a specific phenotype is called “linkage analysis.” By elucidating the association with susceptibility genes for a disease, it is possible to clarify where the gene is located in the HLA class II region, in particular. Identifying the genomic location of a particular gene is called “mapping.” The mapping is performed by accumulating information on the frequency of each genotype in a population with a hereditary character whose association with the gene is to be analyzed and by revealing the relationship between the hereditary character and the genotype.

Each genotype frequency in a population is herein designated as “allele frequency.” In general, when the frequency of a genotype is significantly high in a population with a particular hereditary character, it can be assumed that the microsatellite corresponding to a genotype is located in the genome in the vicinity of the causative gene for the phenotype. The analysis can be carried out, for example, as follows: first, a particular disease is specified for testing, and then microsatellite analysis is carried out to identify the genotype. The same analysis is performed in a group of normal healthy persons. Frequency of the genotype is compared between the two groups. When there is a significant difference in the genotype frequency between the two, then the disease is assumed to be associated with the number of repeat units of a microsatellite representing the genotype. Further, mode of inheritance of the susceptibility genes for the disease can be estimated by retrospective pedigree analysis for the association of the disease with the genotype.

The above-mentioned microsatellites are located in the HLA class II region. Genes playing important roles in the immunological system have been mapped in the HLA class II region. Many disease-associated genes previously reported have also been mapped in this region. Thus, microsatellites located in the HLA class II region and giving enough PIC are useful markers for linkage analysis for a variety of hereditary characters. By using the 21 microsatellites disclosed in the present invention, the HLA class II region with an overall length of about 1.1. Mb is examined at average resolution of 52 Kb by linkage analysis. The present invention is greatly significant providing microsatellites enabling such high-resolution analysis of the HLA class II region, where important information is believed to be contained densely.

The present invention is illustrated in more detail with reference to following EXAMPLES, but is not to be construed as being limited thereto.

### EXAMPLE 1

### Detection of Microsatellite Repeats in the HLA Class II Region

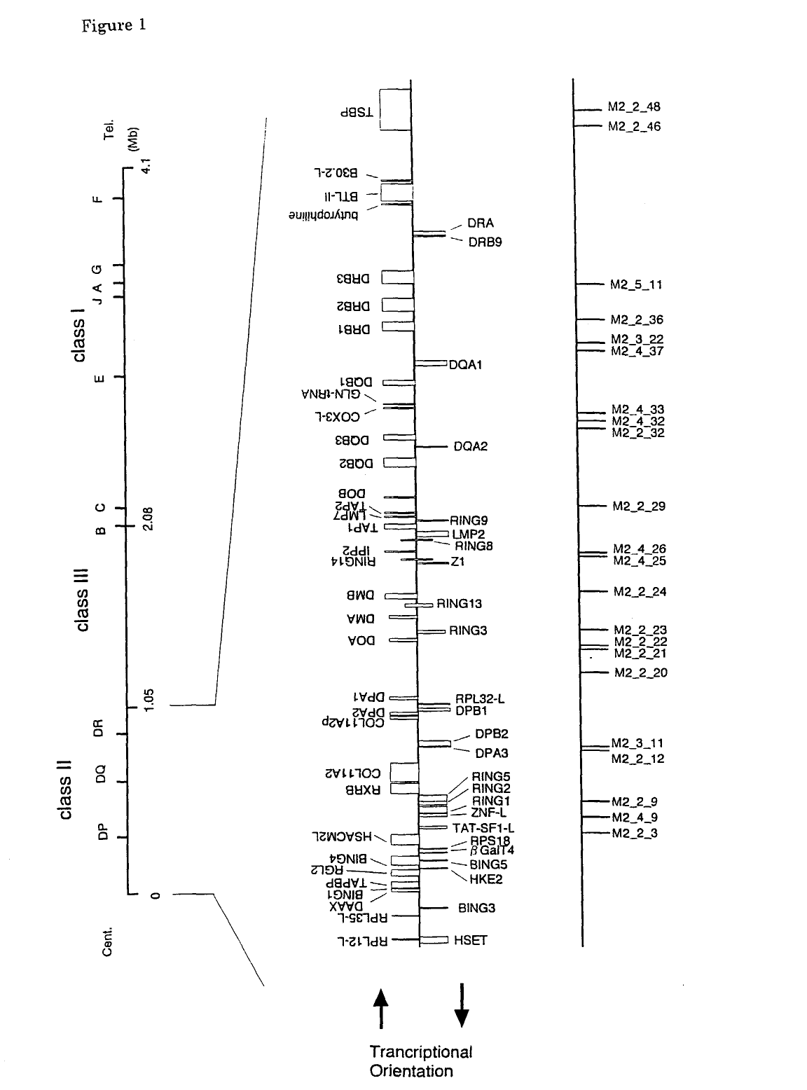

The entire sequence of the HLA class II region from the HSET to TSBP genes (FIG. 1) (The MHC sequencing consortium (1999) Nature 401, 921-923) was retrieved from the database (http://www.sanger.ac.uk/HGP/Chr6/MHC.shtml). To detect microsatellites with di- to penta-nucleotide repeats in this approximately 1.1 Mb region, the genomic sequence was subjected to microsatellite detection analysis by the computer program Sputnik (Abajian, http://www.abajian.com/sputnik/). Of the detected microsatellites, those di-nucleotide repeats carrying more than 10 repeat units and those tri-, tetra-, and penta-nucleotide repeats with over 5 repeat units defined the final selection as these were expected to display a high degree of polymorphism (Weber, W. J. L. (1990) Genomics 7, 524-530).

### EXAMPLE 2

### Identification of Microsatellite Repeats in the HLA Class II Region

Microsatellite repeats identified in the HLA class II region (1.1 Mb from the HSET to TSBP genes, FIG. 1) (The MHC sequencing consortium (1999) Nature 401, 921-923) amounted to 494 in total, consisting of 158 di-, 65 tri-, 163 tetra-, and 108 penta-nucleotide repeats (Table 1). Four tri-nucleotide repeats are localized inside the coding sequences of functional genes. The exon 4 of the Daxx gene included a microsatellite repeat M2_{—}3_{—}3, consisting of (GAG)₅, which encodes polyglutamic acids. Another microsatellite M2_{—}3_{—}4, (GAG)₂GAA(GAG)₃, localized in the exon 1 sequence of the BING1 gene, also encodes polyglutamic acids. The RXRB gene contained M2_{—}3_{—}8, (GCG)₆, which gives rise to polyalanines, in exon 1. The first exon of the COL11A2 gene possessed M2_{—}3_{—}10, (CTC)₄, which encodes polyleucines. Among them, the three microsatellite repeats, M2_{—}3_{—}3, M2_{—}3_{—}4, and M2_{—}3_{—}10, did not exhibit any repeat polymorphism.

TABLE 1
Microsatellites in the HLA class II region

| nucleotide repeat | total | ≧5 repeats | ≧10 repeats | ≧20 nucleotides |
|---|---|---|---|---|
| di | 158 | | 51 | 51 |
| tri | 65 | 28 | | 8 |
| tetra | 163 | 54 | | 54 |
| penta | 108 | 12 | | 27 |
| total | 494 | 94 | 51 | 140 |

145

According to the criteria that microsatellites with more than 10 repeats for di-nucleotide repeats and with more than 5 repeats for tri-, tetra-, and penta-nucleotide repeats are expected to display a high degree of repeat polymorphism (Weber, W. J. L. (1990) Genomics 7, 524-530), 51 di-, 28 tri-, 54 tetra-, and 12 penta-nucleotide repeats (in total, 145) were selected among the total 494 microsatellites contained in the class II region. These are summarized in Table 1 and include 94 perfect repeats, 46 imperfect repeats, and five compound repeats (Table 2). The bulk of these microsatellite consisted of perfect repeats as compound repeat sequences were relatively rare.

TABLE 2
Repeat units of 145 microsatellites in the HLA class II region

| nucleotide repeat | perfect | | imperfect | | compound | | total | |
|---|---|---|---|---|---|---|---|---|
| di | 34 | (13) | 13 | (1) | 4 | (3) | 51 | (17) |
| tri | 23 | (1) | 5 | (1) | 0 | (0) | 28 | (2) |
| tetra | 32 | (6) | 21 | (4) | 1 | (1) | 54 | (11) |
| penta | 5 | (0) | 7 | (1) | 0 | (0) | 12 | (1) |
| total | 94 | (20) | 46 | (7) | 5 | (4) | 145 | (31) |

(): the number of polymorphic microsatellites (see text).

### EXAMPLE 3

### Isolation of Human Genomic DNA

A total of 190 unrelated healthy Japanese blood donor volunteers were enrolled in the present invention. Genomic DNAs were isolated from lymphoblastoid cell lines or peripheral blood leukocytes by phenol extraction after lysis with proteinase K and 0.5% sodium dodecyl sulfate (SDS) (Inoko, H. et al. (1986) Hum. Immunol. 16, 304-312).

### EXAMPLE 4

### Detection of Microsatellite Polymorphism

Out of those 145 microsatellite repeats, 41 repeats were randomly chosen and investigated as to the degree of repeat polymorphism. To roughly survey the degree of repeat polymorphism of these microsatellite repeats, the size of PCR amplified products was investigated by the fluorescent-based genotyping method using human genomic DNAs derived from 8 Japanese using HLA homozygous B-cells lines.

The fluorescent-based genotyping method is as follows. Fluorescent dye-conjugated PCR primers were unilaterally labeled at the 5′-end with the fluorescent reagent, 6-FAM, HEX, TET, or NED (PE biosystems Japan Co. and GENSET SA). PCR amplification of microsatellites was carried out in a 20 μl PCR reaction containing 2 μl of dNTP (2.5 mM each), genomic DNA (5 μl; 2 ng/μl), 2 μl of 10×buffer (100 mM Tris-HCl, pH 8.3, 500 mM KCl, and 15 mM MgCl₂), 20 pmol of forward and reverse primers, and 0.5 U TaKaRa recombinant Taq polymerase (Takara Shuzo Co.) in an automated thermal cycler (PE biosystems Japan Co.). PCR reaction conditions were as follows: after initial denaturation for 5 min at 96° C., annealing for 1 min at 56° C., and extension for 1 min at 56° C., amplifications were processed through 30 temperature cycles consisting of 45-sec denaturation at 96° C., 45-sec annealing at 56° C., and 1-min extension at 72° C. with a final extension of 7 min for 72° C. Each PCR product was denatured for 5 min at 96° C., pooled, mixed with formamide-containing loading buffer, and then separated on 4% polyacrylamide denaturing gels containing 8 M urea with a size standard marker of GS500 TAMURA (PE biosystems Japan Co.) using an ABI 377 automated sequencer XL.

Thirty-one of the above-mentioned 41 microsatellite repeats (76%) were predicted to be quite polymorphic in the Japanese population by a rough survey using 8 Japanese HLA homozygous B-cells lines (Table 2).

### EXAMPLE 5

### Estimation of Allele Frequencies of Microsatellite Repeats

To examine allele frequencies of these 31 polymorphic microsatellite repeats by direct counting, 21 of them were subjected to fluorescent-based genotyping using genomic DNAs from 190 normal Japanese healthy blood donor volunteers. The PCR reaction was carried out in a 96-well plate and PCR products were run with a size standard marker of GS500 ROX (PE biosystems Japan Co) using an ABI 3700 automated sequencer. Other conditions were the same as described in EXAMPLE 4.

Observed heterozygosity, expected heterozygosity, and the polymorphism information content (PIC) value, which are contingent on the number of alleles in samples and the sample size, were calculated from the observed frequencies in the population. Observed heterozygosity was calculated with:

Ht(Obs)=Hn/Wn,

Where Hn is the number of individuals that show heterozygosity at a given microsatellite locus, and Wn is the total number of individuals whose alleles at a given locus were examined, expected heterozygosity (Ht) was calculated with:

Ht=1Σpᵢ²

and PIC was calculated with:

PIC=Ht−ΣΣ2pᵢ²pⱼ²

where pᵢ and pⱼ are the observed frequencies of i th and j th alleles at a given microsatellite locus.

Information on these 21 markers, including localization, repeat unit, allele number, and size range as well as heterozygosity values, PIC, and amplification PCR primers, are listed in Table 3. Heterozygosity was in the range of 0.03 to 0.94 with an average of 0.58. The number of alleles ranged from 2 to 28 with an average of 11.38. The PIC value was between 0.03 and 0.94 with 0.57 on average. These 21 new polymorphic microsatellite markers are almost uniformly interspersed, approximately every 49 kb on average within the HLA class II region (FIG. 1).

TABLE 3
Characteristics of microsatellite markers

| | | | | | | |
|---|---|---|---|---|---|---|
| | | | | HT | | |
| | | | No. of | (Exp) | | |
| Micro- | | Repeat unit ^{c} | alleles^{d} | HT | | |
| satel- | | Original allel | Range | (Obs)^{e} | SEQ | |
| lite^{a} | Localization^{b} | Clone | (bp) | PIC | ID | PCR primers |

| | | | | | | |
|---|---|---|---|---|---|---|
| M2_4_9 | Tel. (20kb)/ARE1- | (TTCC)3(TTCCC)2(TTCC)8 | 9 | 0.58 | 1 | TTCC: GGGATTGATTCCAAAACCC |
| | hom | allele 465 | 369-475 | 0.45 | 2 | GGAA: GAGATCAAGACCATCCTGGC |
| | Cen. (18kb)/RING1 | dJ1033B10 | | 0.56 | | |
| M2_2_9 | IN: RING2 | (TC)13 | 6 | 0.57 | 3 | TC: TGTTTGCCAGGAACTGTGC |
| | | allele 363 | 359-371 | 0.58 | 4 | GA: ACTATGCAGCATCCAAGGC |
| | | dJ1033B10 | | 0.51 | | |
| M2_2_12 | Tel. (15kb)/COLIA2 | (TA)14 | 17 | 0.79 | 5 | TA: TTGCAAATACGATGTCGAAGG |
| | Cen. (63kb)/DPB1 | allele 465 | 455-499 | 0.80 | 6 | AT: AAACCTCCTAACCTCTGTGACC |
| | | dJ1033B10 | | 0.77 | | |
| M2_3_11 | Tel. (21kb)/COLIA2 | (TCT)4T3(TCT)4 | 14 | 0.77 | 7 | TCT: GAGGTCAGCTTCCTCAAATAGG |
| | Cen. (57kb)/DPB1 | allele 429 | 423-454 | 0.24 | 8 | AGA: CCCACACCTGTAATCTTAGTGC |
| | | dJ1033B10 | | 0.75 | | |
| M2_2_20 | Tel. (41kb)/DPA1 | (A)33(TA)15 | 15 | 0.89 | 9 | TA: CCACTCCCATCTTATAGTTGTGTC |
| | Cen. (18kb)/DNA | allele 408 | 393-435 | 0.35 | 10 | AT: AATTCCATTCGCCCAGAG |
| | | 019A | | 0.88 | | |
| M2_2_21 | Tel. (7kb)/DNA | (AT)25 | 15 | 0.79 | 11 | AT: CCAATGTTTGATAGCAGACTGG |
| | Cen. (18kb)/RING3 | allele 339 | 292-336 | 0.43 | 12 | TA: CCTAGAGATTCCTCCGTATTAGTTC |
| | | 014 | | 0.77 | | |
| M2_2_22 | Tel. (11kb)/DNA | (GT)14 | 10 | 0.81 | 13 | GT: GGAGACACATTCAAACCATAGC |
| | Cen. (14kb)/RING3 | allele 205 | 197-219 | 0.78 | 14 | AC: CAATTGGTGACATACATCAACTTG |
| | | 014 | | 0.78 | | |
| M2_2_23 | IN: RING3 | (CA)13 | 5 | 0.76 | 15 | CA: TTGCATACACTCTGAAGCAGC |
| | | allele 293 | 289-297 | 0.73 | 16 | TG: TCCCTGTGGATGTCAAGAATC |
| | | 027 | | 0.72 | | |
| M2_2_24 | Tel. (2kb)/DMB | (GT)11 | 3 | 0.26 | 17 | GT: GAATGGATGCTGCATGAGG |
| | Cen. (73kb)/LMP2 | allele 437 | 433-437 | 0.28 | 18 | AC: AAGTGTTGAAGGAACTCCCTGC |
| | | HA14-III802 | | 0.24 | | |
| M2_4_25 | Tel. (49kb)/DMB | (TATC)12 | 7 | 0.69 | 19 | TATC: TCACTCATGGTTGCTTTTCC |
| | Cen. (27kb)/LMP2 | allele 201 | 189-213 | 0.70 | 20 | GATA: GAATGATAGGAGTCCATTGTGG |
| | | HA14-III802 | | 0.64 | | |
| M2_4_26 | Tel. (50kb)/DMB | (AATA)6 | 4 | 0.24 | 21 | AATA: TTGTGGTTTCAGCTACTCAGG |
| | Cen. (25kb)/LMP2 | allele 183 | 183-200 | 0.25 | 22 | TATT: TTCTTTCATTTGGCCTCTACTG |
| | | HA14-III802 | | 0.22 | | |
| M2_2_29 | Tel. (7kb)/TAP2 | (TC)4TT(TC)6 | 2 | 0.03 | 23 | TC: TACCTTATCATTACCGGAATGC |
| | Cen. (4kb)/DOB | allele 380 | 370-380 | 0.03 | 24 | GA: CGCTGGACCAGAAAGTTAGG |
| | | HA14-III802 | | 0.03 | | |
| M2_2_32 | Tel. (97kb)/DOB | (AT)6AC(AT)5(GT)5 | 5 | 0.50 | 25 | AT: GGCAGCAGAATGAGACTCTG |
| | Cen. (50kb)/DQB1 | allele 160 | 154-164 | 0.47 | 26 | AT: ACGTCCCATGAGGACAGG |
| | | DV19F1121 | | 0.47 | | |
| M2_4_32 | Tel. (21kb)/DQB3 | (GAAG)10 | 9 | 0.47 | 27 | GAAG: GTTCTGGAGATCTGTGGTGG |
| | Cen. (62kb)/DQA1 | allele 380 | 318-396 | 0.83 | 28 | CTTC: GGACTCCAGTTTCAATGCC |
| | | F1121 | | 0.78 | | |
| M2_4_33 | Tel. (114kb)/DOB | (TTTA)11 | 11 | 0.77 | 29 | TTTA: TCATTATCCCCAGTTCAATGAC |
| | Cen. (33kb)/DQB1 | allele 267 | 237-279 | 0.52 | 30 | TAAA: GGGACAGAGCGAGACTCTG |
| | | p797a11 | | 0.74 | | |
| M2_4_37 | Tel. (18kb)/DQA1 | (TTTG)2TTCG(TTTG)2 | 2 | 0.09 | 31 | TTTC: AATGAGGTAATATAGGAAGCAGTGG |
| | Cen. (26kb)/DRB1 | allele 408 | 406-408 | 0.05 | 32 | GAAA: TTTGTTCCTGGTCTCGCTC |
| | | dJ93N13 | | 0.09 | | |
| M2_3_22 | Tel. (34kb)/DQA1 | (TTA)5 | 3 | 0.05 | 33 | TTA: TGCACATAGAGAGCTCCAATC |
| | Cen. (16kb)/DRB1 | allele 174 | 172-181 | 0.01 | 34 | ATT: AGGCAGGAGGTTTGCTTG |
| | | dJ93N13 | | 0.05 | | |
| M2_2_36 | IN:DRB1 | (GT)17 | 28 | 0.95 | 35 | GT: ACTGCAGACACAACTACGGG |
| | | allele 212 | 200-258 | 0.45 | 36 | AC: TCCTTGCTCAGGATAGAGAGG |
| | | dJ93N13 | | 0.94 | | |
| M2_5_11 | Tel. (49kb)/DRB1 | (TTCTT)3T4(TTCTT)TCT(TTCTT) | 6 | 0.78 | 37 | TTCTT: CCAGATTTCCTAGATTACCATCATC |
| | Cen. (3kb)/DRB3 | allele 318 | 285-318 | 0.57 | 38 | AAGAA: TGAAATTGCAACCAGAATATCAC |
| | | dJ172K2 | | 0.74 | | |
| M2_2_46 | IN:TSBP | (TC)10 | 5 | 0.69 | 39 | TC: AGATGGATTCACCTATTGTTCG |
| | | allele 392 | 385-394 | 0.67 | 40 | GA: TCATCACTTGCCAACCTCC |
| | | dJ1077I5 | | 0.63 | | |
| M2_2_48 | IN:TSBP | (TC)26 | 9 | 0.62 | 41 | TC: ATCCCTAACCCTCACGCC |
| | | allele 243 | 221-251 | 0.54 | 42 | GA: GGTGTGGACAACTTTAGTGGC |
| | | dJ1077I5 | | 0.56 | | |

^{a} Naming of microsatellite markers consists of three parts; firstly M2 represents the name of temporary consensus genomic sequence, subsequent_2,_3,_4 and _5 indicate the numbers of nucleotides in repetition units, and the last part represents serial numbers. A total of 21 markers are listed.
^{b} Detailed locations are given according to the genome sequence in this region (The MHC sequencing consortium (1999) Nature 401, 92 1-923). The most adjacent telomeric (Tel.) and centromeric (Cen.) gene names, and their distances (kb) from each marker are indicated.
^{c} Under repeat units, the sizes of original alleles in the genomic sequences determined from cosmid, PAC, or BAC clones are given. At the bottom, the names of cosmid, PAC, or BAC clones are indicated.
^{d} A total number of alleles detected in the present invention is given. Range (bp) indicates the size of range of all alleles at each micro satellite locus.
^{e} HT(Exp), HT(Obs), and PIC represent expected heterozygosity, observed heterozygosity, and PIC (polymorphism information content), respectively.

What is claimed is:

1. An oligonucleotide primer, wherein said primer is capable of specifically hybridizing to a DNA having the sequence of the flanking regions of a microsatellite selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48.

2. The oligonucleotide primer according to claim 1, wherein the sequence of said primer is selected from the group consisting of SEQ ID NOs: 1-42.

3. A kit for determining the number of repeat units of a microsatellite selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48, the kit comprising a pair of oligonucleotide primers having the sequence of the flanking regions of said microsatellite.

4. The kit according to claim 3, wherein the pair of oligonucleotide primers is selected from the group consisting of
(a) SEQ ID NO: 1 and SEQ ID NO: 2,
(b) SEQ ID NO: 3 and SEQ ID NO: 4,
(c) SEQ ID NO: 5 and SEQ ID NO: 6,
(d) SEQ ID NO: 7 and SEQ ID NO: 8,
(e) SEQ ID NO: 9 and SEQ ID NO: 10,
(f) SEQ ID NO: 11 and SEQ ID NO: 12,
(g) SEQ ID NO: 13 and SEQ ID NO: 14,
(h) SEQ ID NO: 15 and SEQ ID NO: 16,
(i) SEQ ID NO: 17 and SEQ ID NO: 18,
(j) SEQ ID NO: 19 and SEQ ID NO: 20,
(k) SEQ ID NO: 21 and SEQ ID NO: 22,
(l) SEQ ID NO: 23 and SEQ ID NO: 24,
(m) SEQ ID NO: 25 and SEQ ID NO: 26,
(n) SEQ ID NO: 27 and SEQ ID NO: 28,
(o) SEQ ID NO: 29 and SEQ ID NO: 30,
(p) SEQ ID NO: 31 and SEQ ID NO: 32,
(q) SEQ ID NO: 33 and SEQ ID NO: 34,
(r) SEQ ID NO: 35 and SEQ ID NO: 36,
(s) SEQ ID NO: 37 and SEQ ID NO: 38,
(t) SEQ ID NO: 39 and SEQ ID NO: 40, and
(u) SEQ ID NO: 41 and SEQ ID NO: 42.

5. A method for determining the number of repeat units of a microsatellite, the method comprising a step for determining the number of repeat units in the region of which DNA can be amplified by using a pair of oligonucleotide primers selected from the group consisting of,
(a) SEQ ID NO: 1 and SEQ ID NO: 2,
(b) SEQ ID NO: 3 and SEQ ID NO: 4,
(c) SEQ ID NO: 5 and SEQ ID NO: 6,
(d) SEQ ID NO: 7 and SEQ ID NO: 8,
(e) SEQ ID NO: 9 and SEQ ID NO: 10,
(f) SEQ ID NO: 11 and SEQ ID NO: 12,
(g) SEQ ID NO: 13 and SEQ ID NO: 14,
(h) SEQ ID NO: 15 and SEQ ID NO: 16,
(i) SEQ ID NO: 17 and SEQ ID NO: 18,
(j) SEQ ID NO: 19 and SEQ ID NO: 20,
(k) SEQ ID NO: 21 and SEQ ID NO: 22,
(l) SEQ ID NO: 23 and SEQ ID NO: 24,
(m) SEQ ID NO: 25 and SEQ ID NO: 26,
(n) SEQ ID NO: 27 and SEQ ID NO: 28,
(o) SEQ ID NO: 29 and SEQ ID NO: 30,
(p) SEQ ID NO: 31 and SEQ ID NO: 32,
(q) SEQ ID NO: 33 and SEQ ID NO: 34,
(r) SEQ ID NO: 35 and SEQ ID NO: 36,
(s) SEQ ID NO: 37 and SEQ ID NO: 38,
(t) SEQ ID NO: 39 and SEQ ID NO: 40, and
(u) SEQ ID NO: 41 and SEQ ID NO: 42.

6. A method for mapping of susceptibility genes for disease associated with HLA class II alleles, by using a microsatellite marker selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48, the method comprising:
(a) determining the number of repeat units of said microsatellite,
(b) estimating the allele frequencies of patients and controls, based on said number, and
(c) comparing the allele frequencies of patients with those of controls.

7. The method according to claim 6, the method comprising:
(a) amplifying a region of microsatellite using the oligonucleotide primer capable of selectively hybridizing to a DNA having a sequence of flanking regions of said microsatellite,
(b) determining the number of repeat units of said microsatellite,
(c) estimating the allele frequencies of patients and controls, based on the number, and
(d) comparing the allele frequencies of patients with those of controls.

8. A method for genotyping of a microsatellite allele selected from the group consisting of M2_{—}4_{—}9, M2_{—}2_{—}9, M2_{—}2_{—}12, M2_{—}3_{—}11, M2_{—}2_{—}20, M2_{—}2_{—}21, M2_{—}2_{—}22, M2_{—}2_{—}23, M2_{—}2_{—}24, M2_{—}4_{—}25, M2_{—}4_{—}26, M2_{—}2_{—}29, M2_{—}2_{—}32, M2_{—}4_{—}32, M2_{—}4_{—}33, M2_{—}4_{—}37, M2_{—}3_{—}22, M2_{—}2_{—}36, M2_{—}5_{—}11, M2_{—}2_{—}46, and M2_{—}2_{—}48, the method comprising:
(a) amplifying a region of the microsatellite, and
(b) determining the number of repeat units of said microsatellite.

9. The method according to claim 7, wherein said amplifying is performed by using the oligonucleotide primer selected from the group consisting of SEQ ID NOs: 1-42.
